(19) United States
(12) Patent Application Publication
(10) Pub. No.: US 2026/0245133 A1
(43) Pub. Date: Aug. 20, 2026

(54) METHOD AND SYSTEM FOR AUTOMATICALLY CREATING A USER PROFILE

(71) Applicant: Miele & Cie. KG, Nordrhein-Westfalen (DE)

(72) Inventors: Daniel CARSTEN, Bielefeld (DE); Maksim FRESE, Paderborn (DE); Lukas KÖPP, Rheda-Wiedenbrück (DE)

(21) Appl. No.: 19/538,398

(22) Filed: Feb. 12, 2026

(30) Foreign Application Priority Data
Feb. 15, 2025 (BE) 2025/0014

(51) Int. Cl. G06Q 30/0601; G16H 20/60

(52) U.S. Cl. CPC G06Q 30/0631; G16H 20/60

(57) ABSTRACT

A method and a system for automatically creating a user profile for the use of a kitchen appliance and for providing an assistance function, the method comprising the steps of: providing usage information about the use of the kitchen appliance and/or about a user of the kitchen appliance and/or about foods that are prepared with the kitchen appliance; creating the user profile by a software module agent using the provided user information; and providing the assistance function using the created user profile.

### CROSS-REFERENCE TO RELATED APPLICATION

This application claims priority pursuant to 35 U.S.C. 119 to Belgium Application No. 2025/0014, filed Feb. 12, 2025, which application is incorporated herein by reference in its entirety.

### FIELD OF THE INVENTION

The invention relates to a method and a system for automatically creating a user profile.

### BACKGROUND OF THE INVENTION

In the prior art, there are various approaches that deal with the collection and management of information on nutritional habits as well as dealing with the use of recipes and automatic programs for kitchen appliances. For example, there are applications that make it possible for a user to keep food diaries or shopping lists. Users can manually enter meals, recipes, or required ingredients there. Apps are also available for shopping and ordering groceries, allowing users to select or deselect ingredients, and sending instructions or special requests to delivery services. In restaurants, it is also possible to inform the staff directly of specific food preferences or intolerances in order to make appropriate adjustments to the dishes.

For kitchen appliance manufacturers, the development of automatic programs is a time-consuming and resource-intensive process. To provide such programs, manufacturers must develop recipes, test them with their own equipment, and validate the necessary parameters before the programs can be made available to customers. Although there are already supplier-provided automatic programs, some of which can be loaded onto household appliances via an update, solutions that make the automatic creation of individual user profiles possible are still lacking. Likewise, there are no known applications that allow the sharing of such user profiles with others in order to take preferences, dislikes, intolerances or allergies into account in a collaborative manner.

Current solutions typically require active input from the user and manual data maintenance, which can be time-consuming and impractical, in particular for food diaries and user profiles. Automated systems that collect data from a networked kitchen, integrate the data into a user profile, and use the data for various purposes such as filtering large recipe collections, providing personalized health advice, or validating new recipes, are not yet available.

### SUMMARY OF THE INVENTION

An object is therefore to specify an improved method and/or an improved system.

According to the invention, this object is achieved by a method having the features of claim 1. In addition, the object is achieved by a system having the features of claim 14. Advantageous embodiments and developments of the invention can be found in the following dependent claims.

Accordingly, a method for automatically creating a user profile for the use of a kitchen appliance and for providing an assistance function is proposed. The method includes the following steps: providing usage information about the use of the kitchen appliance and/or about a user of the kitchen appliance and/or about food being prepared with the kitchen appliance; creation of the user profile by a software module agent using the provided user information; and providing the assistance function using the created user profile.

Furthermore, a system is proposed for automatically creating a user profile for the use of a kitchen appliance, and for providing an assistance function. The system comprises the kitchen appliance, a computing module for executing a software module agent, and a providing module. The providing module is designed to provide usage information about the use of the kitchen appliance and/or about a user of the kitchen appliance and/or about food being prepared with the kitchen appliance. The computing module is designed to create the user profile by means of the software module agent using the provided user information. The providing module is also designed to provide the assistance function using the created user profile.

A user profile for the use of a kitchen appliance preferably includes personal and/or user-specific data that are collected and/or stored for the individual adaptation and optimization of the use of the kitchen appliance. A user profile for the use of a kitchen appliance may preferably contain information about a user's nutritional habits, preferences, dislikes, intolerances and/or allergies. Furthermore, a user profile for the use of a kitchen appliance can include information about frequently used recipes, preferred preparation methods and/or appliance settings. A user profile for the use of a kitchen appliance can still be used to give recommendations, filter new recipes, or personalize automatic programs.

Usage information relating to the use of the kitchen appliance and/or to a user of the kitchen appliance and/or to food preferably includes data relating to the user's interaction with the kitchen appliance and/or the food used. Usage information relating to the use of the kitchen appliance and/or a user of the kitchen appliance and/or food may include, for example, the frequency and type of use of the appliance. Similarly, usage information about the use of the kitchen appliance and/or about a user of the kitchen appliance and/or about food may contain information about preferred or avoided foods. Furthermore, usage information about the use of the kitchen appliance and/or about a user of the kitchen appliance and/or about food can be used to create personalized recommendations or to adapt the functionality of the appliance to the individual needs of the user. Usage information about the use of the kitchen appliance and/or about a user of the kitchen appliance and/or about food can also form a basis for the automatic creation of user profiles.

In particular, until now there has been a lack of options for sharing automatically generated profiles with others or for saving and validating recipe changes and individual adjustments in a cooking community. However, this greatly simplifies the development of new, validated recipes for kitchen appliances and enriches the use of such appliances through an expanded range of recipes and programs.

Creating user profiles for different users offers the advantage of making planning and/or preparation easier for specific individuals and/or for a larger group of people. Discussing and taking into account food preferences, aversions and/or allergies, which can otherwise prove challenging, is significantly simplified by the present method. Furthermore, nutrition-conscious users benefit from the fact that by providing their user profile to restaurants or delivery services, their user-specific nutritional needs can be addressed. Furthermore, household appliance manufacturers can benefit from such user profiles, because sharing and/or validating personal or external data, such as recipes, allows new automatic programs for frequently used recipes to be learned and made available to other users quickly and easily. This can save costs for the creation of such automated programs.

The present method allows, for example, a user profile to be automatically created in the course of the user's everyday life, detailing his food preferences, dislikes, and intolerances, without requiring the user to take time to do so. The creation of the user profile occurs automatically and in the background without the user being obviously aware of this. The user profile can preferably also be dynamically and continuously expanded in order to automatically recognize changes and/or adjustments over time, such as changed eating habits. The user profile created in this way can form the basis for an assistance system, which can then for example provide recipe suggestions and/or nutritional advice. Furthermore, the user profile can be shared with other users, so that for example these user profiles can be taken into account when creating menus and/or preparing dishes. In addition, new automatic programs for the kitchen appliance can be created quickly and easily via a community system using the user profiles.

For example, a personalized user profile can be created in an existing system. Information is preferably stored automatically in this user profile. This information preferably includes cooked meals, recipes, automatic programs and/or additional information provided by the user. Furthermore, the process and/or system can be supplemented by a food inventory management system, used for example to store information about food that is stored or is currently available. Furthermore, user information can also be supplemented with image data of food. Such image data can be captured, for example, from a refrigerator and/or food storage room. In addition, it is possible to provide information about food purchases as additional user information.

A user profile can thus be derived from all the collected information. For example, from recipes, frequently used foods can be saved as user preferences. Unused food items are preferably stored as “unconsumed food”. The user may also be actively prompted to indicate food preferences and dislikes in order to determine whether some foods are foods that happen to have never been used, or are instead disliked foods, foods that are not tolerated, and/or foods to which there are allergies. Of course, such information can also be entered manually by the user via a user interface in order to in this way optimize from the outset the creation of the user profile.

In another aspect, it is proposed that the provided assistance function, using the created user profile, assists the user in selecting food for the preparation of a dish and/or in purchasing food and/or in selecting a recipe to be prepared with the kitchen appliance and/or in adapting a recipe and/or in carrying out food preparation and/or in complying with dietary needs and/or in receiving nutritional advice.

During a selection of foods for preparing a dish the assistance function can give the user recommendations based on individual preferences, intolerances or available ingredients. The assistance function can support the user when shopping for food by generating shopping lists or suggesting alternative products. The assistance function can help the user choose a recipe to prepare with the kitchen appliance by filtering recipes according to taste, nutritional goals or available ingredients. The assistance function can guide the user in adapting a recipe by making suggestions for modifications regarding portion size, calorie content, or alternative ingredients. The assistance function can support the user in carrying out food preparation by providing step-by-step instructions, automation options, or adjustments to appliance settings. The assistance function can support the user in meeting dietary needs by analyzing and evaluating recipes in relation to health requirements and personal nutritional goals. The assistance function can accompany the user during nutritional counseling by providing individual recommendations for a balanced diet based on collected usage information.

Based on the user profile created, recipe recommendations can be tailored to the user, for example. For example, a traffic light system can be used to indicate to the user whether a recipe contains only foods that the user has already consumed or whether the recipe also includes foods that the user has not consumed before, and therefore could potentially be a case of a dislike, intolerance, and/or allergy.

In another aspect, it is proposed that the provided assistance function, using the created user profile, shares at least parts of the usage information and/or the user profile with other users via a public platform and/or makes this visible to other users in order to assist the user and/or other users of the public platform in selecting food and/or recipes and/or to adapt recipes provided via the public platform based on the user's created user profile and/or on the user profiles of the other users.

The assistance function provided preferably shares at least parts of the usage information and/or user profile with other users via a public platform in order to make exchange and networking between users possible. The provided assistance function makes information that is preferably relevant to other users visible in order to facilitate personalized recommendations, collaborative learning or the adaptation of recipes. The provided assistance function preferably makes possible a targeted selection of the information to be shared, in order to take into account data protection and individual user preferences. The assistance function provided can preferably be controlled via sharing options, so that the user himself determines which content is visible to other users. The provided assistance function preferably supports the collaborative development of new recipes or kitchen appliance applications by making possible the exchange and validation of user experiences. The assistance function provided preferably uses the shared information to continuously improve the quality and variety of recipe suggestions or assistance functions.

The user profile can therefore preferably be used to share it with other users within a community platform. For example, if friends are invited for a meal, the shared user profiles can be used to make a comparison with recipes that are to be cooked. The user profiles do not need to be displayed in full, but can, purely by way of example, show the host, by using a traffic light system, whether a recipe is suitable for the guests, or whether there might be problems with certain foods. It is also not necessary here to fully disclose for which guest or food item the problems occur, in order to ensure anonymity. In this way, the host can easily select a recipe that seems to suit all user profiles. Such a recipe can also be automatically suggested from a database by comparing user profiles. This saves the user time.

The same principle can also be used for restaurants and delivery services, preferably on the basis of the user profile, to check whether problems might arise when using certain foods. This information is not always sufficiently transparent for the guest or customer, or a menu item may be selected incorrectly. Restaurants and delivery services can in this way adapt a menu to suit an individual customer. This has significant advantages, in particular in the field of gourmet cuisine.

In another aspect, it is proposed that recipes be recognized in a plurality of user profiles carried out by a predetermined number of users of the public platform.

This allows, for example, the identification of recipes that have been prepared multiple times by multiple users and thus may attract a good deal of attention. Temporary food trends or general preferences, for example, can also be decisive factors. The detection of such recipes is preferably achieved through comparison algorithms that examine the majority of user profiles for such recipes. Similarity comparisons from the field of natural language processing, analytical comparison methods, and also machine learning methods such as large language models, can be used to carry out such comparisons.

In another aspect, it is proposed that the identified recipes be analyzed by a machine learning model, particularly a generative one, in order to extract parameters and/or information from the recipes.

The system preferably collects information about recipes cooked by the user. These can be recipes from various online sources, such as Chefkoch.de or other recipe websites, or from offline sources such as cookbooks and/or magazines. The user can also digitize offline recipes, for example by taking a picture, and provide them. Users can provide online recipes by specifying or clicking on a link. A generative machine learning model, such as a transformer model, is then preferably used to analyze the recipe in question and, based on this, to extract important information from the recipe, for example about parameters, food and/or preparation methods, etc. Furthermore, web crawling can also be used to directly access online sources. The platform Chefkoch.de, for example, is well-known and includes many favorite recipes that have been highly rated by many users. For certain basic dishes, such as lasagna, there is a recipe that is frequently used by users and is cooked again and again. Such recipes can be automatically analyzed here.

In another aspect, it is proposed that the software module agent creates an automatic program for the kitchen appliance from the recognized or analyzed recipes, which program can be executed by the kitchen appliance.

The software module agent preferably recognizes or analyzes recipes and generates an automatic program from them that is specifically designed for execution by the kitchen appliance. The software module agent preferably creates the automatic program on the basis of the recipe contents, the required preparation steps, and the specific appliance settings. The software module agent preferably optimizes the automatic program by adapting parameters, such as temperature, cooking time or stirring speed, to the characteristics of the relevant kitchen appliance. The software module agent preferably makes possible the automatic conversion of recipes into executable programs, thereby reducing manual adjustments by the user or manufacturer. The software module agent preferably takes into account usage information and stored preferences in order to tailor the automated program individually to the particular user. The software module agent can preferably convert continuously updated or community-validated recipes into automatic programs, so that new programs can be efficiently provided.

For example, the same external recipe is repeatedly recognized and analyzed. In this process, the parameters used by customers when implementing recipes are analyzed and, if necessary, automatically adapted to the relevant kitchen appliance. Frequently used and/or modified parameters are then preferably stored. If a recipe from an external source is recognized and prepared by the user, a comparison can be made with parameters already used for this recipe by other networked users. Based on this, recommendations can be given, such as: “other users who have cooked this recipe have on average set a temperature that is 10° higher.”

In another aspect, it is proposed that, based on information from the public platform that corresponds at least partially to the usage information and/or user profile, the user is given an adjustment suggestion for adapting a device setting of the kitchen appliance, or that the adjustment of the device setting is made automatically on the kitchen appliance.

Based on information from the public platform that corresponds at least partially to the parts of the usage information and/or of the user profile, the user is preferably given an adjustment suggestion for adapting an appliance setting of the kitchen appliance. The user is preferably offered recommendations for optimizing the appliance settings in order to enable personalized preparation according to individual preferences or the collected experiences of other users. The user can preferably have the appliance settings on the kitchen appliance adjusted automatically, so that optimal preparation is ensured without manual intervention. Users are preferably provided with configurable options to determine whether adaptation suggestions should be manually confirmed or implemented directly. The user is preferably enabled to benefit from collectively gathered knowledge by receiving device settings adapted for specific recipes or preparation methods. Users are preferably given the opportunity to save their own adaptations and share them with the platform, so that future adaptation suggestions can be further improved.

In another aspect, it is proposed that the user provide, by user feedback via the public platform, an evaluation of an automatic program and/or of a setting of the automatic program of the kitchen appliance, on the basis of which an adjustment of the automatic program is made.

Furthermore, user feedback can for example also be analyzed to determine whether the parameters used have led to a successful result. In this way, a new and validated automatic program for a new recipe is developed, in particular step by step. Another example is a private recipe made available to the general community, including the appliance parameters used for the kitchen appliance. These can also be automatically shared with the community platform via the networked kitchen appliance. Other kitchen appliance users can then for example recreate this private recipe using the same parameters, or have them automatically downloaded and executed by the networked kitchen appliance. If the results are successful, the user preferably gives positive feedback about the recipe on the community platform. The system can then preferably evaluate this feedback and thus validate the parameters used for such a private recipe. An automatic program for the kitchen appliance can also be developed in this way. The kitchen appliance can then preferably suggest these parameters to the user who wants to recreate this recipe. This allows users to preferably become experts within the community and advance within a tiered system. Entered private recipes, self-entered feedback, and/or feedback received on recipes are evaluated and taken into account for this purpose, and can thus regulate the credibility and the inclusion of new automated programs.

In another aspect, it is proposed that the assistance function provided is trained to communicate with a mobile application by using the created user profile, in order to assist the user with instructions via the mobile application.

On the basis of the user profile and as desired by the user, the present method can also provide nutritional advice and/or assistance with food selection. This can preferably be freely adjustable by the user. This can include, for example, recipe suggestions and/or nutritional advice, such as information as to which foods should be used more often or less often. Push notifications can also be sent in everyday life that indicate a targeted food intake.

In a further aspect, it is proposed that the usage information comprises information about food and/or prepared food and/or a recipe and/or a recipe result and/or an automatic program selected on the kitchen appliance for preparing the food and/or an appliance setting of the kitchen appliance and/or image data and/or text data of the food and/or the recipe and/or stored food and/or food purchases and/or manual user information.

Furthermore, additional metadata can also serve as usage information, so the list should not be understood as restrictive.

In another aspect, it is proposed that at least some of the usage information could be acquired by using augmented reality data glasses.

Augmented reality data glasses are preferably a wearable, glasses-like electronic device that displays digital information in the user's field of vision, overlaying it with the real environment. Augmented reality data glasses preferably make it possible to display virtual elements, such as instructions, hints or interactive operating elements, while the user continues to perceive his surroundings. Augmented reality data glasses can preferably contain sensors, cameras and motion detection systems to dynamically adapt content to the user's viewing direction and environment. Augmented reality data glasses are preferably used to support work processes, navigation, information display, or interaction with smart devices. Augmented reality data glasses can preferably be connected to the kitchen appliance to display preparation steps, recipe information or operating instructions directly in the user's field of vision. Augmented reality data glasses can preferably be operated via voice control, gestures and/or eye movements to make possible hands-free interaction. Augmented reality glasses can preferably display individually adaptable content based on the user's preferences and usage information. Furthermore, user information can also be supplemented with data streams from user assistance systems, such as AR data glasses. For example, a camera can capture a food purchase and/or a food storage area and/or a cooking process and provide these image data as additional user information. Preferably, information is extracted from these data that can be added to the user profile to continuously enrich it.

In another aspect, it is proposed that the creation of the user profile by a software module agent includes identifying and/or authenticating the user.

Identifying and/or authenticating the user preferably includes methods for uniquely identifying a person in order to provide personalized functions or secure access. User identification and/or authentication can preferably be achieved through biometric features such as facial recognition, fingerprint scanning, or iris scanning. User identification and/or authentication can preferably be done via digital access data such as user name and password, or via two-factor authentication with additional security mechanisms. User identification and/or authentication can preferably be supported by smart devices, wearable sensors or mobile devices that enable automatic recognition. Identifying and/or authenticating the user can preferably be used for personalized settings, access to stored user profiles, or security-related functions of a device. Identifying and/or authenticating the user can preferably be used in networked households to control kitchen appliances in order to take individual preferences into account or to restrict access to certain functions. The user profile can be created automatically, for example after user identification. This makes possible a one-time identification of the user on the kitchen appliance, e.g. by profile selection, and/or by automatic identification by means of a camera system. User identification can also be made possible, supported by Bluetooth and/or ultra-wideband tagging. In other words, identification and/or authentication can involve facial recognition and/or speech recognition and/or proximity detection of the user.

In another aspect, it is proposed that the computing module and/or the providing module be each designed as software modules in a server or cloud service environment, and the kitchen appliance be communicatively connected to the server or cloud service environment.

The computing module and/or the providing module are preferably designed as software modules in a server or cloud service environment to make possible a centralized processing and provision of data. The computing module and/or the providing module preferably run computationally intensive processes in a networked infrastructure in order to optimize the performance and scalability of the data processing. The computing module and/or the providing module are preferably designed to operate independently of local devices and to be accessible via an internet connection. The computing module and/or the providing module preferably make possible a secure storage and processing of usage information as well as the provision of personalized assistance functions. The computing module and/or the providing module are preferably integrated into a cloud service environment in order to make possible a flexible, location-independent use and synchronization with various end devices. The computing module and/or the providing module can preferably be dynamically adapted to changing requirements through scalable resources in order to ensure efficient data processing and rapid provision of information.

The invention also includes a computer program product comprising instructions which, when the method is executed by a computer, cause the computer to execute the method.

The invention also includes a computer-readable medium on which the computer program product is stored.

The kitchen appliance is preferably an oven or a stove or a microwave or an air fryer or a steamer or a multicooker or a sous-vide appliance or a rice cooker or an induction hob or a coffee machine or a kettle or a food processor or an ice cream machine.

### BRIEF DESCRIPTION OF THE DRAWINGS

An exemplary embodiment of the invention is shown purely schematically in the drawings and is described in more detail below. Shown are:

FIG. 1 shows a schematic representation of an embodiment of a present system; and

FIG. 2 shows a schematic flowchart of an embodiment of the present method.

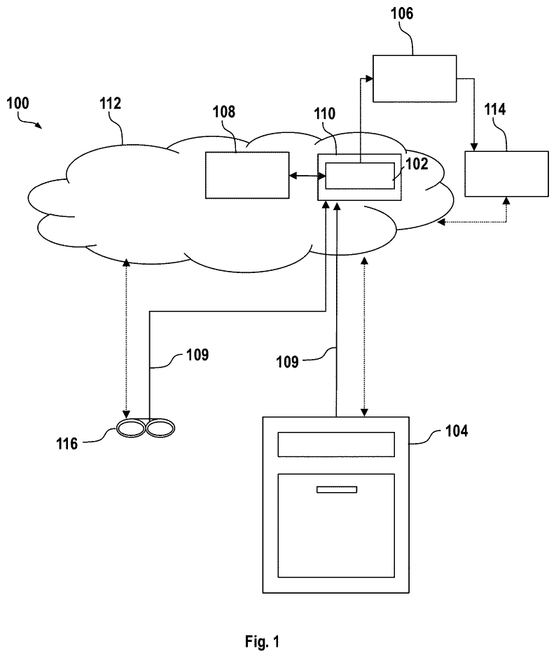

FIG. 1 shows a schematic representation of an embodiment of a system 100 according to the present invention. The system 100 is designed to automatically create a user profile 102 for the use of a kitchen appliance 104 and to provide an assistance function 106.

For this purpose, the system 100 includes the kitchen appliance 104, a computing module 108 for executing a software module agent, and a providing module 110. The kitchen appliance 104 is in the present case an oven. The kitchen appliance 104 can however also be a stove or a microwave or an air fryer or a steamer or a multicooker or a sous-vide device or a rice cooker or an induction hob or a coffee machine or a kettle or a food processor or an ice cream machine.

The providing module 110 is designed to provide usage information 109 about the use of the kitchen appliance 104 and/or a user of the kitchen appliance 104 and/or food being prepared with the kitchen appliance 104. The computing module 108 is designed to create the user profile 102 by means of the software module agent using the provided user information, the providing module 110 being designed to provide the assistance function 106 using the created user profile 102.

The usage information 109 can for example contain information about food and/or food to be prepared and/or a recipe and/or a recipe result and/or an automatic program selected on the kitchen appliance 104 for preparing the food and/or an appliance setting of the kitchen appliance 104 and/or image data and/or text data of the food and/or the recipe and/or stored food and/or food purchases and/or manual user information.

The computing module 108 and the providing module 110 are each designed as software modules in a server or cloud service environment 112. The kitchen appliance 104 is also communicatively connected to the server or cloud service environment 112.

The provided assistance function 106 assists the user, by using the created user profile 102, in selecting food for the preparation of a dish and/or in shopping for food and/or in selecting a recipe to be prepared with the kitchen appliance 104 and/or in adapting a recipe and/or in carrying out food preparation and/or in complying with dietary needs and/or in receiving nutritional advice.

Furthermore, the assistance function 106 uses the created user profile 102 to share at least parts of the usage information 109 and/or of the user profile 102 with other users via a public platform 114 and/or to make it visible to other users in order to assist the user and/or the other users of the public platform 114 in selecting food and/or recipes and/or to adapt recipes provided via the public platform 114 on the basis of the user's created user profile 102 and/or on the basis of user profiles of the other users.

On the basis of information from the public platform 114, which corresponds at least partially to the parts of the usage information 109 and/or the user profile 102, the user can be given an adjustment suggestion for adjusting a device setting of the kitchen appliance 104, or the adjustment of the device setting can be done automatically at the kitchen appliance 104. Furthermore, the user can, through user feedback via the public platform 114, provide an evaluation of an automatic program and/or a setting of the automatic program of the kitchen appliance 104, on the basis of which an adjustment of the automatic program is made.

The system can in addition include augmented reality data glasses 116, through which at least a part of the usage information 109 can be acquired. The augmented reality data glasses 116 are also communicatively connected to the server or cloud service environment 112.

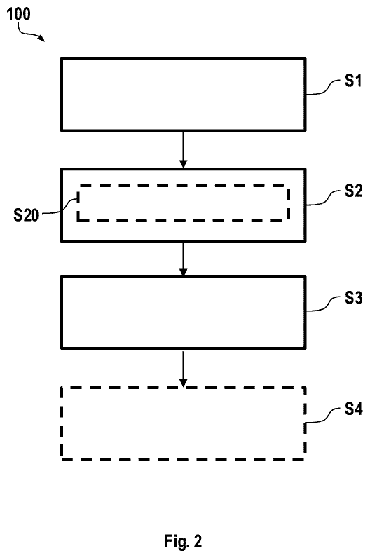

FIG. 2 shows a schematic flowchart of an exemplary embodiment of the method. In a step S1, usage information 109 is provided for the use of the kitchen appliance 104 and/or about a user of the kitchen appliance 104 and/or foods that are prepared with the kitchen appliance 104. In a step S2, a software module agent creates the user profile 102 using the user information provided. In a step S3, the assistance function is provided using the created user profile 102.

The creation S2 of the user profile 102 by a software module agent can optionally include an identification and/or authentication S20 of the user. The optional identification and/or authentication S20 can in turn include facial recognition and/or speech recognition and/or proximity detection of the user.

In addition, the method can optionally include a recognition S4, in a plurality of user profiles, of recipes carried out by a predetermined number of users of the public platform 114. The recognized recipes are analyzed by a machine learning model, in particular a generative one, in order to extract parameters and/or information from the recipes. The software module agent creates an automatic program for the kitchen appliance 104 from the recognized or analyzed recipes, which program can be executed by the kitchen appliance 104.

### List of Reference Signs

100 System
102 User profile
104 Kitchen appliance
106 Assistance function
108 Computing module
109 Usage information
110 Providing module
112 Server or cloud service environment
114 Public platform
116 Augmented reality data glasses
S1 Method step
S2 Method step
S20 Method step
S3 Method step
S4 Method step

1. A method for automatically creating a user profile for the use of a kitchen appliance and for providing an assistance function, the method comprising the steps:
providing usage information for the use of the kitchen appliance and/or about a user of the kitchen appliance and/or about foods that are prepared with the kitchen appliance;
creation of the user profile by a software module agent using the user information provided; and,
providing the assistance function by using the created user profile.

2. The method according to claim 1, wherein the provided assistance function using the created user profile assists the user in selecting food for the preparation of a dish and/or in purchasing food and/or in selecting a recipe to be prepared with the kitchen appliance and/or in adapting a recipe and/or in carrying out food preparation and/or in complying with dietary needs and/or in receiving nutritional advice.

3. The method according to claim 1, wherein the provided assistance function uses the created user profile to share at least parts of the usage information and/or of the user profile with other users via a public platform and/or to make it visible to other users in order to assist the user and/or the other users of the public platform in selecting food and/or recipes and/or to adapt recipes provided via the public platform on the basis of the user's created user profile and/or on the basis of user profiles of the other users.

4. The method according to claim 3, wherein in addition a recognition takes place of such recipes in a plurality of user profiles that are carried out by a predetermined number of users of the public platform.

5. The method according to claim 4, wherein the recognized recipes are analyzed by an in particular generative machine learning model in order to extract parameters and/or information from the recipes.

6. The method according to claim 4, wherein the software module agent creates an automatic program for the kitchen appliance from the recognized or analyzed recipes, which program can be executed by the kitchen appliance.

7. The method according to claim 3, wherein, on the basis of information from the public platform which corresponds at least partially to the parts of the usage information and/or the user profile, the user is given an adaptation suggestion for adjusting an appliance setting of the kitchen appliance, or the adjustment of the appliance setting is carried out automatically at the kitchen appliance.

8. The method according to claim 3, wherein via user feedback via the public platform, the user provides an evaluation of an automatic program and/or of a setting of the automatic program of the kitchen appliance, on the basis of which an adjustment of the automatic program is made.

9. The method according to claim 1, wherein the provided assistance function is configured, using the created user profile, to communicate with a mobile application in order to assist the user with instructions via the mobile application.

10. The method according to claim 1, wherein the usage information comprises information about food and/or about food to be prepared and/or about a recipe and/or about a recipe result and/or about an automatic program selected on the kitchen appliance for preparing the food and/or about an appliance setting of the kitchen appliance and/or about image data and/or text data of the food and/or about the recipe and/or about stored food and/or about food purchases and/or manual user information.

11. The method according to claim 1, wherein at least part of the usage information can be acquired by using a pair of augmented reality data glasses.

12. The method according to claim 1, wherein the creation of the user profile by a software module agent includes an identification and/or an authentication of the user.

13. The method according to claim 12, wherein the identification and/or authentication comprises facial recognition and/or speech recognition and/or proximity recognition of the user.

14. A system for automatically creating a user profile for using a kitchen appliance and for providing an assistance function, the system comprising the kitchen appliance, a computing module for executing a software module agent and a providing module, wherein the providing module is configured to provide usage information about the use of the kitchen appliance and/or about a user of the kitchen appliance and/or about food prepared with the kitchen appliance, wherein the computing module is configured to create the user profile by means of the software module agent using the provided user information, and wherein the providing module is configured to provide the assistance function using the created user profile.

15. The system according to claim 14, wherein the computing module and/or the providing module are each configured as software modules in a server or cloud service environment, and wherein the kitchen appliance is communicatively connected to the server or cloud service environment.
